(19) United States
(12) Patent Application Publication
(10) Pub. No.: US 2002/0081266 A1
(43) Pub. Date: Jun. 27, 2002

(54) Spray dried powders for pulmonary or nasal administration

(75) Inventors: Austen John Woolfe, North Weald (GB); Xian Ming Zing, Grays (GB); Alan Langford, Hoddesdon (GB)

Correspondence Address: BEYER WEAVER & THOMAS LLP, P.O. BOX 778, BERKELEY, CA, 94704-0778, US

(73) Assignee: Norton Healthcare Ltd.

(21) Appl. No.: 09/930,109

(22) Filed: Aug. 14, 2001

Related U.S. Application Data
(63) Continuation-in-part of application No. 09/643,145, filed on Aug. 21, 2000, now abandoned.
(60) Provisional application No. 60/150,095, filed on Aug. 20, 1999.

(51) Int. Cl. A61K 9/14; A61K 31/57

(52) U.S. Cl. 424/46; 514/179

(57) ABSTRACT

A formulation for pulmonary or nasal administration comprising a mixture of particles of two or more drugs or excipients produced by spray drying and suitable for administration without further processing of the particles.

### CROSS-REFERENCE TO RELATED APPLICATIONS

This application is a continuation-in-part of U.S. patent application Ser. No. 09/643,145, entitled COMBINATION FORMULATION, filed Aug. 21, 2000, which application claims priority from U.S. Provisional Patent Application No. 60/150,095, entitled COMBINATION FORMULATION, filed Aug. 19, 1999. Priority is claimed from these applications and their disclosures are incorporated by reference herein for all purposes.

### BACKGROUND OF THE INVENTION

1. Field of the Invention

The present invention relates to a process for preparing combination pharmaceutical formulations for pulmonary or nasal administration. The invention also relates to formulations for such uses. The invention relates particularly to a combination of a drug and at lest one other component. The component or components may be an excipient designed to stabilise the drug or give better content uniformity of the drug or product. The invention relates particularly to combinations of drugs used for the treatment of asthma.

2. Description of Related Art

Asthma can be categorised in a number of stages according to official guidelines, e.g., British Thoracic Society (Thorax; 1997; 52 (suppl. 5) 51-528); Canadian Thoracic Society (Can Med Assoc. J; 1992; 147: 420-8); American Thoracic Society (Am J Respir Crit Care Med; 1995; 152 (suppl) 577-5120). In these guidelines regimens are suggested for treatment of symptoms of increasing severity. These normally start with a β₂ agonist or antimuscarinic agent and then add a steroid if the symptoms are not well enough controlled. This means that many patients have to carry two or even three inhalers with the different types of drug. Combination products have found wide commercial acceptability and a number are widely marketed. Others have been proposed in the patent literature.

Examples of β₂ agonists are salbutamol, rimiterol, bambuterol, fenoterol, pirbuterol, isoetharine and terbutaline. Recently, long acting β₂ agonists have been introduced e.g. salmeterol, eformoterol (sometimes known as formoterol). Examples of antimuscarinic agents include ipatropium bromide and oxitropium bromide. Examples of steroids include beclomethasone esters, fluticasone, budesonide and mometasone.

Examples of combination products include:

a) Short acting β₂ agonist+antimuscarinic, e.g., salbutamol+ipatropium bromide (Duovent®) fenoterol+ipatropium bromide (Combivent®).

b) Short acting β₂ agonist+corticosteroid e.g. salbutamol+beclomethasone (Ventide®).

c) Long acting β₂ agonist+corticosteroid e.g. salmeterol+fluticasone EP (Seretide®) eformoterol+budesonide EP

Such products can be used normally as aerosols, either for delivery into the lung or nose, i.e., as metered dose inhalers, as dry powder inhalers usually for pulmonary use, as pressurised pump solutions for nasal administration or by the use of nebulizers.

If the formulation is a solution then there are few problems with uniformity of dosage apart from those normally associated with such devices, e.g., valve design and actuator design. However, if the product is formulated as a suspension there are more problems, for example settling of the suspension in the aerosol over time, caking on the sides of the aerosol container or non uniformity of the mixture in dry powder devices. These problems are exacerbated by the fact that the powders have to be a controlled particle size to ensure delivery to the place of action. For example, in inhalation aerosols the particle size is normally controlled to a mass mean diameter of 1-5 microns.

The problems of non-uniformity are particularly pronounced when one of the drugs is given in a low dosage or there is some form of interaction or non compatibility between the two active ingredients in suspension.

Problems of low dose arise for example with ipatropium bromide because the dose can be as low as 20 micrograms per shot; eformoterol where a common dose is 12 micrograms per shot; and salmeterol where a dose of 25 micrograms is often given.

### SUMMARY OF THE INVENTION

The present invention provides particles produced by spray drying having smooth surfaces. Generally, elliptical or oval particles may be produced, preferably generally spherical particles. Spherical particles confer considerable advantages in ease of formulation and administration.

According to a first aspect to a present invention there is provided a formulation for pulmonary or nasal administration comprising a mixture of particles of two or more drugs or excipients produced by spray drying and suitable for administration without further processing of the particles.

According to a second aspect of the present invention there is provided a method of manufacture of a formulation for pulmonary or nasal administration including a step of making a mixture of particles of two or more drugs or excipients by spray drying without further processing of the particles.

Spray dried particles of mixtures of drugs or excipients in accordance with this invention may have 90% of the particles with a dimension less than 5 μm.

Preferred formulations have over 50% of the particles with a dimension between 1 and 5 μm.

According to third aspect of the present invention there is provided a metered dose dry powder inhaler containing a powder reservoir containing a formulation in accordance with the first aspect of this invention, optionally also containing one or more further drugs or excipients for example a sugar, for example selected from; lactose, mannitol, dextrose, xylitol and trehalose.

According to a fourth aspect of the present invention there is provided a metered dose dry powder inhaler containing generally spherical particles 90% of which have a dimension below 5 μm which have been produced by spray drying of a drug and one or more drugs or excipients, optionally together with one of more further excipients.

According to a fifth aspect of the present invention a capsule for insufflation containing generally spherical particles 90% of which have a dimension below 5 μm and which have been produced by spray drying of a drug and one or more drugs or excipients, optionally together with further excipients for inhalation by humans or other mammals.

According to a sixth aspect of the present invention there is provided a unit dose pocket or blister package containing generally spherical particles 90% of which have a dimension is below 5 μm and which have been produced by spray drying of a drug and one or more drugs or excipients, optionally together with further excipients.

According to a seventh aspect of the invention a metered dose inhaler contains a CFC propellant, preferably P134a, P227 or a mixture thereof and a suspended drug or drugs where at least one of the drugs is in the form of generally spherical particles 90% of which have a dimension below 5 μm and which have been produced by spray drying without milling or micronization, optionally together with other drugs or excipients.

These and other features and advantages of the present invention will be presented in more detail in the following specification of the invention and the accompanying figures which illustrate by way of example the principles of the invention.

### BRIEF DESCRIPTION OF THE DRAWINGS

FIG. 1 shows scanning electron micrographs of powdered formoterol numerate;

FIG. 2 shows scanning electron micrographs of powdered budesonide;

FIG. 3 shows scanning electron micrographs of a physical mixture of powdered formoterol numerate and budesonide in the ratio 6:100;

FIG. 4 shows scanning electron micrographs of a physical mixture of powdered formoterol numerate and budesonide in the ratio 6:400;

FIG. 5 shows scanning electron micrographs of spray dried budesonide;

FIG. 6 shows scanning electron micrographs of spray dried formoterol numerate and budesonide in the ratio 6:100;

FIG. 7 shows scanning electron micrographs of spray dried formoterol numerate and budesonide in the ratio 6:400.

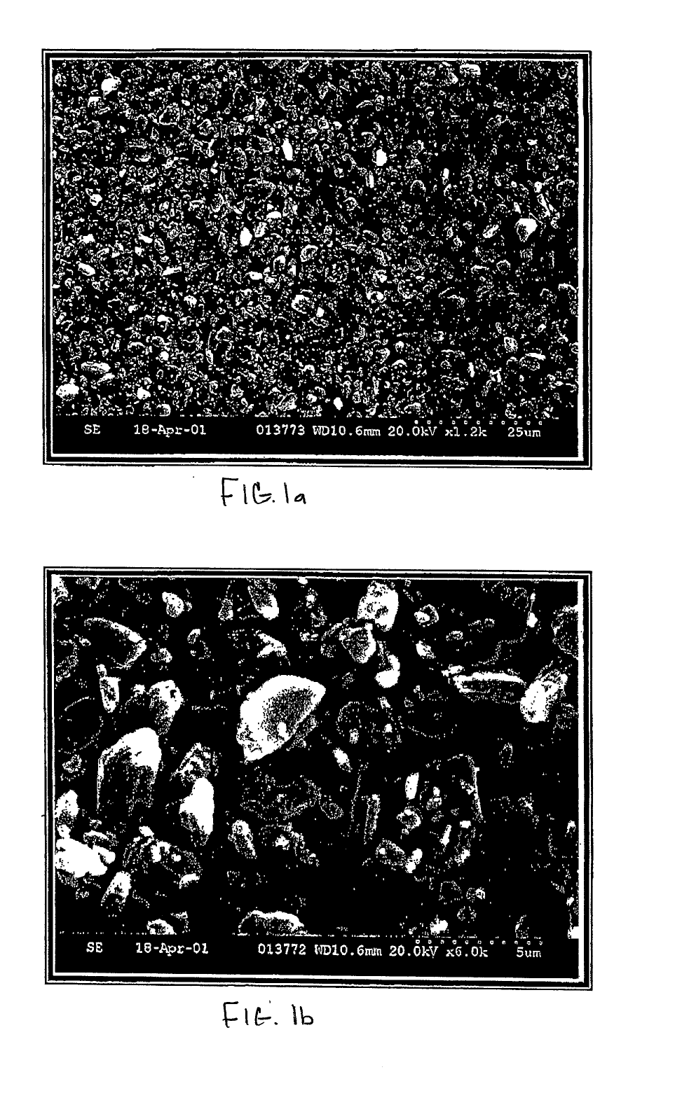

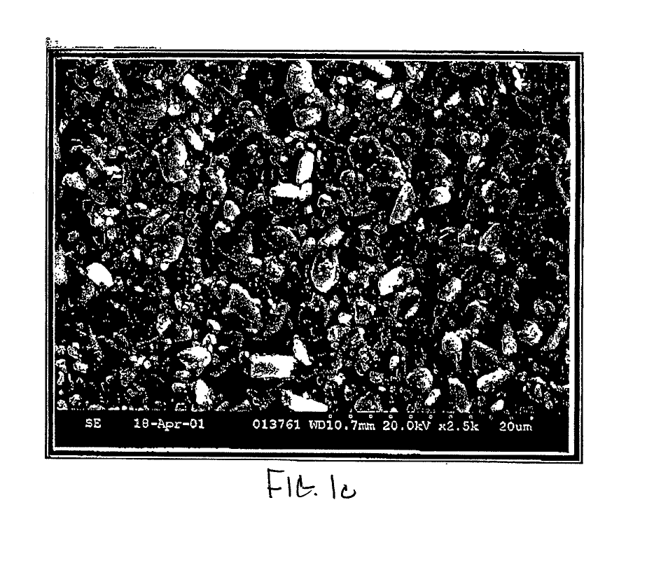

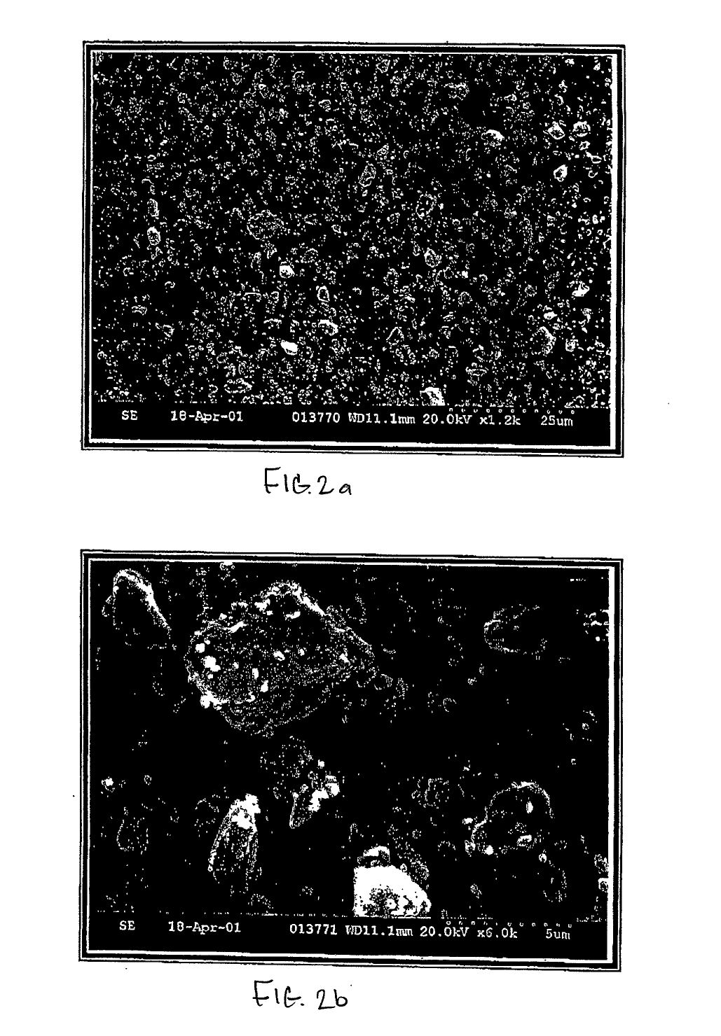

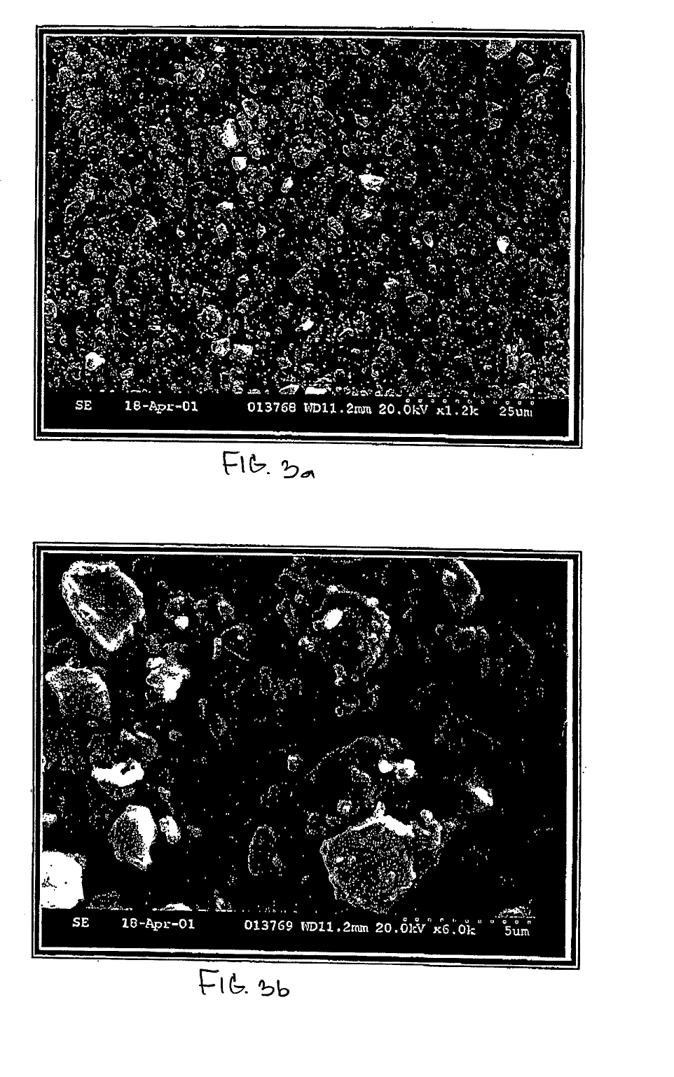

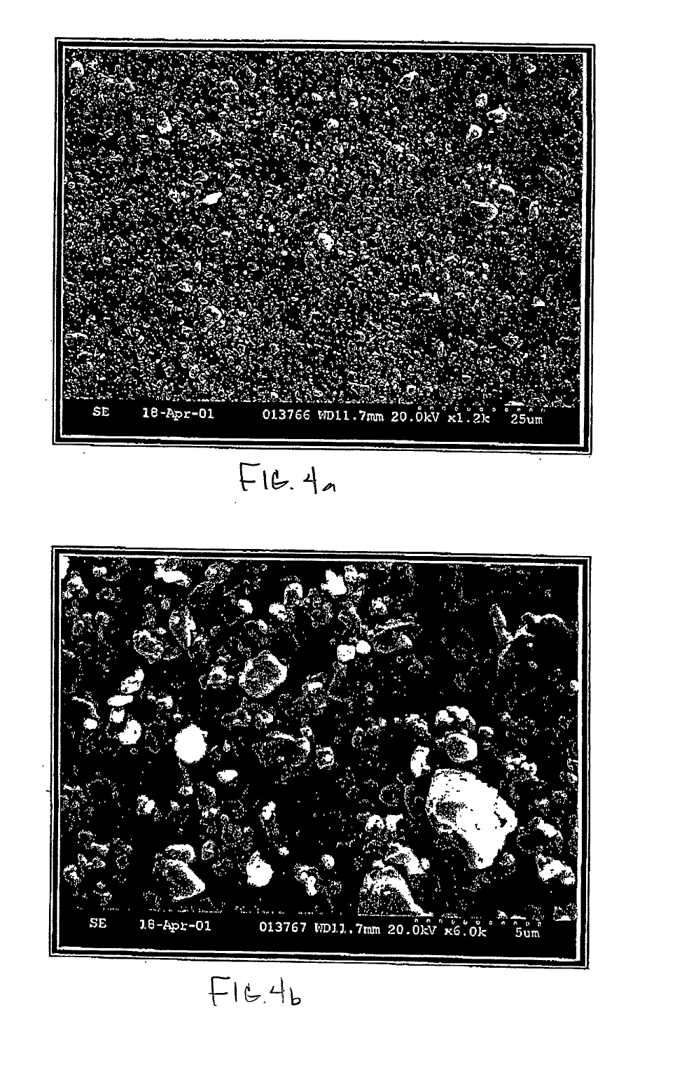

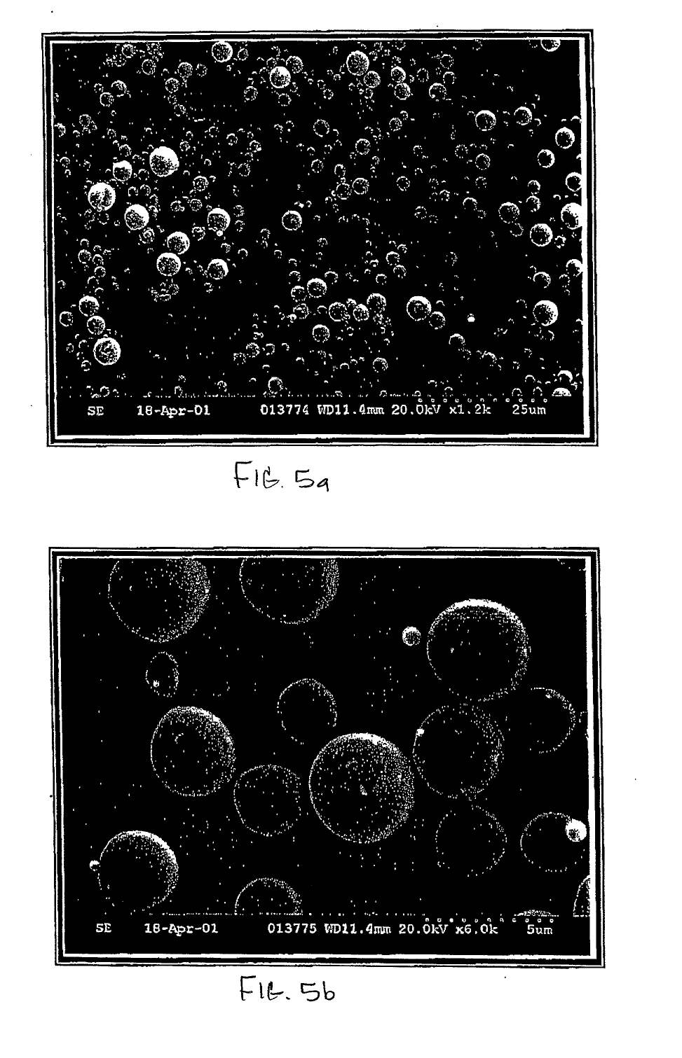

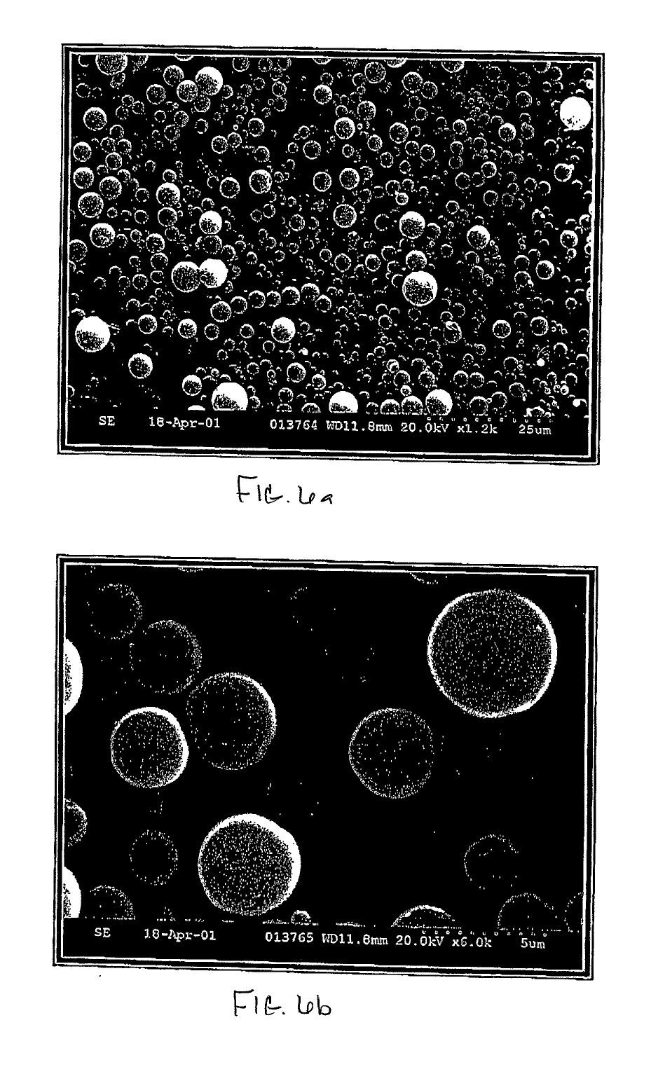

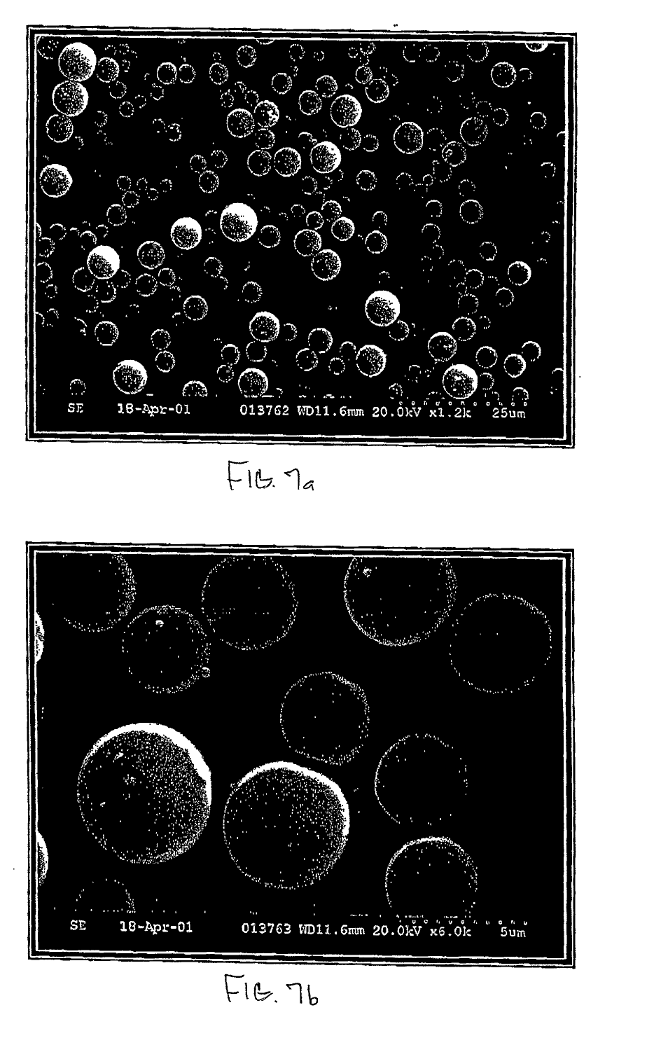

### DETAILED DESCRIPTION OF EMBODIMENTS OF THE INVENTION

Reference will now be made in detail to some specific embodiments of the invention including the best mode contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail in order not to unnecessarily obscure the present invention.

Percentages and amounts used in this specification are by weight unless indicated otherwise.

It has surprisingly been discovered that the particles produced by spray drying in accordance with this invention have smooth surfaces. Generally elliptical or oval particles may be produced preferably generally spherical particles. Spherical particles confer considerable advantages in ease of formulation and administration.

According to a first aspect to a present invention there is provided a formulation for pulmonary or nasal administration comprising a mixture of particles of two or more drugs or excipients produced by spray drying and suitable for administration without further processing of the particles.

According to a second aspect of the present invention there is provided a method of manufacture of a formulation for pulmonary or nasal administration including a step of making a mixture of particles of two or more drugs or excipients by spray drying without further processing of the particles.

Spray dried particles of mixtures of drugs or excipients in accordance with this invention may have 90% of the particles with a dimension less than 5 μm.

Preferred formulations have over 50% of the particles with a dimension between 1 and 5 μm.

Any combination of drugs and excipients including mixtures of drugs and excipients, for example as referred to above may be used. Two or more drugs may be used. These are preferably drugs of same therapeutic class. The preferred examples include two or more selected from; corticosteroids, antimuscarine bronchodilating agents, short acting beta agonists and medium or long acting beta agonists. Preferred cortico steroids include fluticasone, budesonide, beclomethasone and esters thereof. Preferred muscarine bronchial dilating agents include ipatropium, oxitropium, tiotropium, and salts thereof. Preferred short acting beta agonist include salbutamol (albuterol), fenoterol, terbutaline and related compounds including salts thereof. Preferred medium to long acting beta agonist includes formoterol, salmeterol, bambuterol and related compound and salts.

The particles may comprise a single drug or a combination of two or more drugs together with one or more excipients suitable for nasal of pulmonary delivery. Excipients may be sugars, for example selected from; lactose, mannitol, xylitol, trehalose, dextrose and other pharmaceutically acceptable sugars.

Further excipients may include one or more surfactants, preferably a surfactant which is solid at ambient temperatures e.g., 25° C. A carboxylic acid, e.g., oleic acid may be employed. Alternative surfactants include lecithin and sorbitan esters. Desiccant excipients may be employed, preferably an excipient which has a degree of hygroscopicity which is similar to or greater than the drug or drugs in the particles.

Delay release excipients may also be employed to provide for release of a drug over a longer period in vivo in comparison to normal micronised particles of the drug. An excipient of low water solubility may be used, a pharmaceutically acceptable polymer preferably a cellulose derivative, polyvinyl pyrollidone or a sugar derivative.

Further excipients may be selected from pH stabilisers, antioxidants and flavouring agents. These may be chosen if necessary from standard pharmaceutical anti-oxidants, e.g., α-tocopherol or ascorbyl palmitate, or pH modifiers, e.g., citric acid or tris buffer or physiologically acceptable sodium salts, to enable the long term stability of the drug or drugs to be improved over such particles without these excipients.

In preferred embodiments the ratio of one drug or excipient in the particle, is in a ratio greater than 75% or, preferably greater than 85% of the particle compared to another drug in the particle. In an especially preferred embodiment a larger proportion of a drug or excipient is selected to provide superior dose uniformity of the lower proportion drug than may be achieved by conventional mixing with either the second drug or a mixture of the lower dose drug and an excipient carrier.

The larger proportion drug or excipient may be selected to act as a desiccant for the smaller proportion drug to prevent flow problems due to hygroscopicity of the latter. Alternatively or in addition the larger proportion drug or excipient may prevent moisture absorption by the lower proportion drug by acting as a physical barrier due to less of the lower proportion drug being available for moisture absorption at the particle surface.

In a preferred embodiment the higher proportion drug may be a beta agonist and a lower proportion drug may be an antimuscarine bronchodilator.

In an alternative preferred embodiment the higher proportion of drug may be a corticosteroid and the lower proportion drug may be a shorter medium beta acting agonist or an antimuscarine bronchial dilator. In this embodiment a lower dose drug may be selected from formoterol, salmeterol, bambuterol, or salts thereof.

A suitable higher proportion excipient maybe a sugar, preferably selected from lactose, mannitol, dextrose, trehalose, xylitol and sorbitol.

Preferred formulations may also include a stabiliser as an excipient, for example a compound selected to improve the stability of one or more drugs. Such stabilisers include antioxidants for example tocopherols, ascorbyl palmitate, pH modifiers for example citric acid or buffers for example tris buffer.

According to third aspect of the present invention there is provided a metered dose dry powder inhaler containing a powder reservoir containing a formulation in accordance with the first aspect of this invention, optionally also containing one or more further drugs or excipients for example a sugar, for example selected from; lactose, mannitol, dextrose, xylitol and trehalose

According to a fourth aspect of the present invention there is provided a metered dose dry powder inhaler containing generally spherical particles 90% of which have a dimension below 5 μm which have been produced by spray drying of a drug and one or more drugs or excipients, optionally together with one of more further excipients.

An inhaler in accordance with this aspect of invention enables a high respiratory fraction to be delivered to the lungs of a human or other mammal without need for micronisation of the active ingredients.

According to a fifth aspect of the present invention a capsule for insufflation containing generally spherical particles 90% of which have a dimension below 5 μm and which have been produced by spray drying of a drug and one or more drugs or excipients, optionally together with further excipients for inhalation by humans or other mammals.

According to a sixth aspect of the present invention there is provided a unit dose pocket or blister package containing generally spherical particles 90% of which have a dimension is below 5 μm and which have been produced by spray drying of a drug and one or more drugs or excipients, optionally together with further excipients.

A dosage form in accordance with the fourth to sixth aspects of this invention may incorporate an external excipient to improve flow characteristics. Examples of suitable external excipients include lactose, mannitol, trehalose or other sugars or mixtures thereof. A mixture of the same or different materials with different particle sizes may be employed.

According to a seventh aspect of the invention a metered dose inhaler contains a CFC propellant, preferably P134a, P227 or a mixture thereof and a suspended drug or drugs where at least one of the drugs is in the form of generally spherical particles 90% of which have a dimension below 5 μm and which have been produced by spray drying without milling or micronization, optionally together with other drugs or excipients.

### EXAMPLES

The following examples provide additional experimental details relating to methods and compositions in accordance with the present invention. This material intended to assist in an understanding of the present invention and should not be construed to limit the scope of the invention.

### Example 1

Sample Preparation

The following drugs and mixtures were prepared by spray drying using a Buchi 190 MiniSpray Drier:

(i) salbutamol sulphate from aqueous solution: 10 g of salbutamol sulphate was spray dried as a 10% w/v aqueous solution using the spray drying parameters outlined below. These parameters are similar to those used by Chawla, A. et al (International Journal of Pharmaceutics 108 (1994) 233-240):

| | |
|---|---|
| Inlet temperature: | 151-153° C. |
| Outlet temperature: | 75-78° C. |
| Pump setting: | 7 |
| Air flow rate: | 600-700 1 hr⁻¹ |

(ii) salbutamol sulphate from ethanolic solution: 8 g of salbutamol sulphate was dissolved in ethanolic solution for spray drying. The solvent used consisted of ethanol 75%, water 25%. A 0.6% w/v solution was spray dried using the following spray drying parameters:

| | |
|---|---|
| Inlet temperature: | 100-102° C. |
| Outlet temperature: | 60-64° C. |
| Pump setting: | 6 |
| Air flow rate: | 500 1 hr⁻¹ |

(iii) salbutamol from ethanolic solution: salbutamol was spray dried from ethanol (98%) as a 2.5% w/v solution. Initially a solution containing 12.5 g was spray dried. The spray drying parameters used were:

| | |
|---|---|
| Inlet temperature: | 91-94° C. |
| Outlet temperature: | 62° C. |
| Pump setting: | 7 |
| Air flow rate: | 700 1 hr⁻¹ |

The yield was extremely low (6.9%) and material was collected only from the cyclone separator since no powder was present in the collecting vessel.

It was decided to alter the spray drying conditions and hence a lower inlet temperature, lower pump rate and decreased flow rate were used. The second attempt at spray drying Salbutamol BP from ethanolic solution (96%) consisted of 12.5 g of solid spray dried as a 2.5% w/v solution. The spray drying parameters used were:

| | |
|---|---|
| Inlet temperature: | 77-79° C. |
| Outlet temperature: | 48-50° C. |
| Pump setting: | 5 |
| Air flow rate: | 500 1 hr⁻¹ |

The percentage yield was approx 26%.

On this occasion powder was collected from both the collecting vessel and the cyclone.

Salbutamol BP was spray dried again under similar conditions except that the pump setting was increased to 6.9 g of powder was weighed and spray dried as a 2.5% w/v solution from ethanol (96%). The spray drying parameters used were:

| | |
|---|---|
| Inlet temperature: | 77-78° C. |
| Outlet temperature: | 54-56° C. |
| Pump setting: | 6 |
| Air flow rate: | 500 1 hr⁻¹ |

The percentage yield was approx 38%.

(iv) ipratropium bromide from aqueous solution: 5 g of ipratropium bromide was spray dried as a 5% w/v aqueous solution. The spray drying parameters were

| | |
|---|---|
| Inlet temperature: | 151-153° C. |
| Outlet temperature: | 102-104° C. |
| Pump setting: | 7 |
| Air flow rate: | 700 1 hr⁻¹ |

(v) ipratropium bromide from ethanolic solution: ipratropium bromide was spray dried from an ethanolic solution (96%). 10 g in total was spray dried as a 2.5% w/v solution. The spray drying parameters were:

| | |
|---|---|
| Inlet temperature: | 77-79° C. |
| Outlet temperature: | 55-56° C. |
| Pump setting: | 6 |
| Air flow rate: | 500 1 hr⁻¹ |

Note: Practically no powder was collected from the collecting vessel. The powder appeared sticky initially. On storage under vacuum the following day the powder was observed to no longer be elastic/sticky but quite brittle and dry.

(vi) salbutamol sulphate: ipratropium bromide mixtures:

(a) 10:1 weight ratio, from aqueous solution: This co-spray dried system was prepared by weighting 10 g of salbutamol sulphate and 1 g of ipratropium bromide to give a total of 11 g of solids. This was spray dried as a 5% w/v solution (5% total solids) using the parameters given below.

| | |
|---|---|
| Inlet temperature: | 151-153° C. |
| Outlet temperature: | 100-102° C. |
| Pump setting: | 7 |
| Air flow rate: | 600-700 1 hr⁻¹ |

(b) 5:1 weight ratio, from aqueous solution: This co-spray dried system was prepared by weighing 10 g of salbutamol sulphate and 2 g of ipratropium bromide to give a total of 12 g of solids. This was spray dried as a 5% w/v solution (5% total solids) using the parameters given below.

| | |
|---|---|
| Inlet temperature: | 151-153° C. |
| Outlet temperature: | 99-103° C. |
| Pump setting: | 7 |
| Air flow rate: | 700 1 hr⁻¹ |

(c) 2:1 weight ratio, from aqueous solution: This co-spray dried system was prepared by weighing 10 g of salbutamol sulphate and 5 g of ipratropium bromide to give a total of 15 g of solids. This was spray dried as a 5% w/v solution (5% total solids) using the parameters given below.

| | |
|---|---|
| Inlet temperature: | 151-153° C. |
| Outlet temperature: | 99-100° C. |
| Pump setting: | 7 |
| Air flow rate: | 600-700 1 hr⁻¹ |

After spray drying all samples were stored in a vacuum dessicator at 4° C.

The physical characteristics of the spray-dried compounds and mixtures were determined by xray diffraction (XRD), differential scanning calorimetry (DSC), thermogravimetric analysis (TGA), Fourier transform infrared (FTI) and scanning electron microscopy (SEM).

Preparation of Physical Mixes

Physical mixture of salbutamol sulphate or salbutamol BP and ipratropium bromide were prepared by weighing appropriate quantities of the two materials, loading into 30 g amber glass jars and mixing in a Turbula™ mixer for 5 minutes. The weights taken were: for the 10:1 weight ratio, 1 g salbutamol sulphate or Salbutamol BP and 0.1 g of ipratropium bromide; for the 5:1 weight ratio, 1 g salbutamol sulphate or Salbutamol BP and 0.2 g of ipratropium bromide; and for the 2:1 weight ratio, 1 g salbutamol sulphate or Salbutamol BP and 0.5 g of ipratropium bromide.

Powder X-Ray Diffraction (XRD)

The powder X-Ray Diffractometer used was a Siemens D500 Diffractometer which consist of a DACO MP wide-range goniometer. A 1.00° dispersion slit, a 1.00° anti-scatter slit and a 0.15° receiving slit were used. The Cu anode x-ray tube was operated at 40 kV and 30 mA in combination with a Ni filter to give monochromatic Cu Kα X-rays. All measurements were taken from 5 to 35 on the 2 theta scale at a step size of 0.05°/second.

Differential Scanning Calorimetry (DSC)

The Differential Scanning Calorimeter used was a Mettler Toledo DSC 821^{e}, Mettler Toledo STAR^{e} software Version 5.1 with a Solaris operating system. Samples were placed in open (hermetically sealed aluminium with three vent holes) pan types under nitrogen purge. Sample weights were between 5 and 10 mg. DSC experiments were run generally from 30 to 250 or 350° C. (depending on degradation products) at a heating rate of 10° C./minute. Two DSC scans were obtained from each system.

Thermogravimetric Analysis (TGA)

Thermogravimetric analysis was carried out using a Mettler TG 50 linked to a Mettler MT5 balance. Data was processed using Mettler Toledo STAR software Version 5.1 with a Solaris operating system. Sample weights between 5 and 10 mg were used and analysis carried out under nitrogen purge. The scans were generally run from 30 to 350° C. at a heating rate of 10° C./minute. Two TGA scans were obtained for each system.

Scanning Electron Microscopy (SEM)

The scanning electron microscope used was the Hitachi S-3500N variable pressure scanning electron microscope. Samples were mounted and sputtered with gold spray for SEM.

Fourier Transform Infra-red Spectroscopy (FTIR)

The spectrometer used was a Perkin Elmer Paragon 1000 FTIR. KBr discs were prepared based on 1 mg % sample loading. Discs were prepared by grinding the sample with KBr in an agate mortar and pestle, placing the sample in an evacuable KBr die and applying 8 tons of pressure in a Graseby Specac IR press. Two FTIR spectra were obtained for each system.

Salbutamol sulphate as supplied was a crystalline material by XRD. When spray dried from aqueous solution it was amorphous as evidenced by XRD, The amorphous material was relatively stable on heating. There was no obvious exotherm in the DSC thermogram, reflective of recrystallisation from the glass. The infrared spectrum of the spray dried sample compared to the spectrum of the original material showed a change in the OH region and no match for bands at 1546 and 1244 cm⁻¹ seen in the original spectrum. There was inconsistency in the intensity of some bands between the two spectra. Small spherical particles, typical of amorphous material were observed by SEM. Particle diameters ranged from ˜1 μm to ˜8 μm. The surface of the particles was slightly dimpled.

Spray drying from ethanolic solution also resulted in an amorphous material by XRD. Again the DSC showed no obvious exotherm indicative of recrystallisation. Small spherical particles, typical of amorphous material were observed by SEM. Comparisons of SEMs showed that particles were smaller than those produced from the aqueous solution, with particle diameters less than ˜3 μm. The surface of the particles was slightly dimpled.

Salbutamol as supplied was a crystalline material by XRD. On spray drying from ethanolic solute, the XRD indicated the same crystalline form was present, although some peak intensity differences were evident. As the initial conditions used to spray dry the material resulted in a lower yield, the spray drying conditions were adjusted appropriately to improve the yield. A lower inlet temperature, lower pump rate (2 different settings) and decreased flow rate were used. Three spray dried samples were analysed by DSC. The major peak in the DSC occurred at the same position as the melting endotherm of salbutamol base. An exotherm, typical of the presence of an amorphous material that is physically unstable, occurred before the melting endotherm. The position and size of this peak varied between the three samples. The energy change associated with the exotherm was lower if the DSC was performed the day after spray drying. The exotherm was also at a higher temperature. This suggests that the spray dried material contains some amorphous material which rapidly converted to the crystalline form. The infrared spectrum was a good match to the spectrum of the original material. Rough, irregular shaped particles were observed by SEM, with diameters ranging from less than ˜1 μm to ˜8 or 7 μm.

Ipratropium bromide as supplied was a crystalline material by XRD. The DSC showed a major endotherm with a peak at ˜237° C., which can be attributed to melting. However two further lower temperature overlapping endotherms between 80 and 120° C. were also evident. TGA indicated that these lower temperature endotherms represented 3 to 4% to the total solid mass. This suggested the presence of solvent. When spray dried from aqueous solution the material remained crystalline, although the XRD pattern was somewhat different. The DSC of the spray dried material showed four endothermic events. There were two low temperature endotherms between about 85 and 120° C. The TGA did not detect any mass loss associated with these endotherms and the combined energy change associated with them was ˜4 J/g compared to ˜122 J/g for the ipratropium bromide original raw material. There was another small endothermic peak at ˜208° C. before the large melting endotherm. Rough, irregular shaped particles were observed by SEM, with diameters ranging from about 5 to 20 μm.

When spray dried from ethanolic solution, the XRD was very similar to that of the starting material. The DSC showed two low temperature endothermic peaks as well as a higher melting endotherm. The energy changes associated with the lower temperature endotherms was smaller than that of the low temperature endotherms of the starting material (˜49 J/g versus 122 J/g) and the TGA did not detect any mass loss associated with them. The shape of the endotherms was also somewhat different to hose of the starting material. The spray dried sample in the IR showed some changes in the OH region relative to the original material. Large crystalline particles were evidence under SEM with diameters of the order of 60 μm and larger.

Salbutamol sulphate:ipratropium bromide mixtures on spray drying from aqueous solution gave amorphous materials with physicochemical characterisation (XRD, DSC) similar to the spray dried salbutamol sulphate alone. Both DSC and XRD were similar to those of spray dried salbutamol sulphate. At the three ratios studied, the ipratropium bromide appeared to be dispersed in salbutamol sulphate in an amorphous form.

When the infrared spectrum of the 10:1 systems was compared to the equivalent physical mix, the spray dried sample showed changes in appearance in the OH region. There was no match in the spray dried spectrum for bands at 1087 cm⁻¹, 1031 cm⁻¹ and 1245 cm⁻¹ and there were new bands at 1044 cm⁻¹ and 1002 cm⁻¹ in the spray dried sample.

The infrared spectrum of the 5:1 system showed some differences in the OH region. There was no match in the spray dried spectrum for bands in the mechanical mix at 1087 cm⁻¹, 1031 cm⁻¹ and 978 cm⁻¹ and there were additional bands at 1267 cm⁻¹, 1448 cm⁻¹, 1404 cm⁻¹ and 1734 cm⁻¹ in the spray dried sample.

The infrared spectrum of the 2:1 system showed some differences in the OH region and a change in intensity of some bands when compared to the equivalent physical mix. The spray dried sample showed loss of 1245 cm⁻¹, 1087 cm⁻¹ and 1030 cm⁻¹ bands and showed new bands at 1508 cm⁻¹, 1268 cm⁻¹, 1044 cm⁻¹ and 1003 cm⁻¹. Some other minor inconsistencies were apparent.

SEM showed particles from all three systems prepared to be small and spherical, typical of amorphous material.

The 10:1 sample displayed slightly dimpled particles less than 3 μm in diameter.

The 5:1 systems displayed more significantly dimpled particles, with diameters less than 5 μm.

The 2:1 system displayed smooth spherical particles with diameters less than 7 μm.

The samples were tested for degradation of salbutamol. In the co-spray dried systems the level of degradants was below the acceptable limits.

### Example 2

Spray Drying Methods

Samples were spray dried using Büchi 191 MiniSpray Drier.

(i) Budesonide: 8 g of budesonide was spray dried as a 2.5% w/v ethanolic solution (95% ethanol) using the spray drying parameters outlined below.

Inlet temperature: 78-79° C.

Outlet temperature: 52-54° C.

Pump setting: 30%

Aspirator rate: 100%

Air flow rate: 600 lhr⁻¹

The percentage yield was approximately 61%.

(ii) A formoterol fumarate: budesonide mixture, 6:100 weight ratio, was spray dried as a 1.75% w/v ethanolic solution (95% ethanol). This co-spray dried system was prepared by weighing 0.6 g of formoterol fumarate and 10 g of budesonide to give a total of 10.6 g solids. This was spray dried as a 1.75% w/v solution, since the system was not soluble at a 2.5% w/v, using the parameters given below.

Inlet temperature: 78-79° C.

Outlet temperature: 52-56° C.

Pump setting: 30%

Aspirator rate: 100%

Air flow rate: 600 lh⁻¹

The percentage yield was approximately 68%.

(iii) A formoterol fumarate: budesonide mixture, 6:400 weight ratio, was spray dried as a 2.5% w/v ethanolic solution (95% ethanol). This co-spray dried system was prepared by weighing 0.15 g of formoterol fumarate and 10 g of budesonide to give a total of 10.15 g solids. This was spray dried using the parameters given below.

Inlet temperature: 78-79° C.

Outlet temperature: 52-55° C.

Pump setting: 30%

Aspirator rate: 100%

Air flow rate: 600 lhr⁻¹

The percentage yield was approximately 63%.

Preparation of Physical Mixtures

Physical mixtures of formoterol fumarate and budesonide were prepared by weighing appropriate quantities of the two materials, loading into a glass specimen tube and mixing in a Turbula™ mixer for 5 minutes. The powder was mixed with a spatula manually for 2 minutes after mixing in the Turbula mixer. The weights taken were: for the 6:400 weight ratio, 0.015 g of formoterol fumarate and 1 g of budesonide.

Power X-Ray Diffraction (XRD)

The powder X-Ray Diffractometer used was a Siemens D500 Diffractometer consisting of a DACO MP wide-range goniometer. A 1.00° dispersion slit, a 1.00° anti-scatter slit and a 0.15° receiving slit were used. The Cu anode x-ray tube was operated at 40 kV and 30 mA in combination with a Ni filter to give monochromatic Cu Ka X-rays. All measurements were taken from 5 to 35° on the 2 theta scale at a step size of 0.05°/second.

Thermogravimetric Analysis (TGA)

Thermogravimetric analysis was carried out using a Mettler TG 50 linked to a Mettler MT5 balance. Data was processed using Mettler Toledo STAR software Version 5.1 with a Solaris operating system. Sample weights were between 5 and 10 mg and analysis was carried out under nitrogen purge. The scans were run from 30 to 350° C. at a heating rate of 10° C./minute. Two TGA scans were obtained for each system.

Scanning Electron Microscopy (SEM)

The scanning electron microscope used was the Hitachi S-3500N variable pressure scanning electron microscope. Samples were mounted on aluminium stubs and spluttered with gold spray for SEM.

Fourier Transform Infra-red Spectroscopy (FTIR)

The spectrometer used was a Perkin Elmer Paragon 1000 FTIR. KBr discs were prepared based on 1 mg % sample loading. Discs were prepared by grinding the sample with KBr in an agate mortar and pestle, placing the sample in an evacuable KBr die and applying 8 tons of pressure, in a Graseby Specac IR press. Two FTIR spectra were obtained for each systems.

RESULTS

The Following Results Were Observed.

Formoterol Fumarate

Formoterol fumarate starting material was crystalline as evidenced by XRD. TGA showed weight loss of approximately 4% occurring at 80-100° C. consistent with solvent loss. The second endotherm was consistent with melting of the formoterol fumarate. TGA showed further mass loss occurring from 150° C. onwards which appeared to be due to degradation.

SEM showed irregular shaped particles with average particle diameter of 0.5-5 μm. Formoterol fumarate was not spray dried alone.

Budesonide

Budesonide as supplied was a crystalline material as shown by the powder XRD trace. Spray drying from ethanolic solution resulted in an amorphous product as evidenced by XRD. TGA showed little difference between systems.

FTIR showed no change in the spectrum of the spray dried budesonide when compared to unprocessed budesonide.

SEM showed that spray drying resulted in spherical particles, which had a mainly smooth surface morphology apart from a few imperfections. In comparison to the starting material the particles were more uniform in shape. The average particle diameter for both systems was approximately 0.5-5 μm.

Formoterol Fumarate:Budesonide

Formoterol fumarate:budesonide mixtures on spray drying gave amorphous systems. The 6:100 co-spray dried system showed more crystallinity than the 6:400 co-spray dried system. XRD of the physical mixtures showed peaks indicative of crystalline budesonide but peaks indicative of formoterol fumarate could not be detected due to the low concentrations of formoterol fumarate present.

TGA of the 6:100 physical mixture showed weight loss occurring after 180° C. corresponding to a loss of approximately 2%. A weight loss of approximately 1.3% was seen in the same region for the co-spray dried 6:100 system. No such loss was detected for either of the 6:4000 systems. This loss was probably the beginning of degradation of formoterol fumarate.

FTIR showed no apparent difference between physical mixtures and co-spray dried systems for both weight ratios investigated.

SEM showed the physical mix to consist of irregular shaped particles of roughly 0.5 to 5 μm in diameter. The co-spray dried particles were uniform smooth spheres of approximately 1-5.5 μm in diameter.

### Example 3

Spherical particles 1-5 microns in size and formed directly by spray-drying with salbutamol sulphate 120 parts and ipatropium bromide 20 parts by weight were prepared. The larger proportion of salbutamol acted as an agent to cover the ipatropium bromide and so prevent moisture uptake by the ipatropium bromide. The increased weight of the particle compared to the ipatropium alone gave better content uniformity of the lower dose drug.

The particles were either suspended in a mixture of P134a and/or P227 with a cosolvent (ethanol) or a surfactant as appropriate in a metered dose aerosol inhaler, or were mixed with lactose as a flow aid in a metered dose dry powder inhaler, or used as received from the spray dryer in a capsule for insufflation.

Conclusion

Although the foregoing invention has been described in some detail for purposes of clarity of understanding, those skilled in the art will appreciate that various adaptations and modifications of the just-described specific embodiments can be configured without departing from the scope and spirit of the invention. Therefore, the described embodiments should be taken as illustrative and not restrictive, and the invention should not be limited to the details given herein but should be defined by the following claims and their full scope of equivalents.

It is claimed:

1. A formulation for pulmonary or nasal administration comprising a mixture of particles of two or more drugs or excipients produced by spray drying and suitable for administration without further processing of the particles.

2. A formulation as claimed in claim 1, wherein 90% of the particles have a dimension less than 5 μm.

3. A formulation as claimed in claim 2, wherein over 50% of the particles have a dimension between 1 and 5 μm.

4. A formulation as claimed in claim 1 wherein the particles have smooth surfaces.

5. A formulation as claimed in claim 4, wherein the particles are oval or elliptical in shape.

6. A formulation as claimed in claim 4, wherein the particles are spherical.

7. A formulation as claimed in claim 1 comprising two or more drugs.

8. A formulation as claimed in claim 7, wherein the two drugs are of the same therapeutic class.

9. A formulation as claimed in claim 8, wherein the drugs are of two or more therapeutic classes.

10. A composition as claimed in claim 1, wherein the drugs include two or more of the following: corticosteroids, anti antimuscarine bronchodilating agents, short acting beta agonists and medium or long acting beta agonists.

11. A composition as claimed in claim 10, wherein the corticosteroids are selected from fluticasone, budesonide, beclamethasone and esters thereof.

12. A formulation as claimed in claim 10, wherein the antimuscarine bronchodiliating agents are selected from ipratropium, oxitropium, tiotropium and salts thereof.

13. A formulation as claimed in claim 10, wherein the short acting beta agonists are selected from salbutamol, fenoterol, terbutalene and salts thereof.

14. A formulation as claimed in claim 10, wherein the medium to long acting beta agonists are selected from formoterol, salmeterol, bambuterol and salts thereof.

15. A formulation as claimed claim 1, wherein the particles contain one or more drugs together with an excipient.

16. A formulation as claimed in claim 15, wherein the excipient is suitable for nasal or pulmonary delivery.

17. A formulation as claimed in 16, wherein the excipient is a sugar.

18. A formulation as claimed in claim 16, wherein the sugar is selected from lactose, mannitol, xylitol, trehalose, dextrose or mixtures thereof.

19. A formulation as claimed in claim 1 wherein the excipient is a surfactant.

20. A formulation as claimed in claim 19, wherein the surfactant is solid at 25° C.

21. A formulation as claimed in claim 20, wherein the surfactant is selected from carboxylic acids, preferably selected from: oleic acid, lecithin and sorbitan esters.

22. A formulation as claimed in claim 1, wherein the excipient has desiccant properties.

23. A formulation as claimed in claim 22, wherein the excipient has a degree of hygroscopicity similar to or greater than the drug or drugs in the particles.

24. A formulation as claimed in claim 1, wherein the excipient is a delay release agent.

25. A formulation as claimed in claim 24, wherein the excipient has low water solubility.

26. A formulation as claimed in claim 25, wherein the excipient is a pharmaceutically acceptable polymer.

27. A formulation as claimed in claim 1, wherein the ratio of one drug or excipient in the particle to another drug in the particle is greater than 75%.

28. A formulation as claimed in claim 27, wherein the ratio is greater than 85%.

29. A formulation as claimed in claim 27, wherein the larger proportion of drug or excipient is selected to provide superior dose uniformity of the lower proportion drug.

30. A formulation as claimed in claim 27, wherein the larger proportion of drug or excipient may act as a desiccant for the smaller proportion drug.

31. A formulation as claimed in claim 27, wherein the larger proportion drug or excipient prevents moisture absorption by the lower proportion drug.

32. A formulation as claimed in claim 27, wherein the higher proportion drug is a beta agonist and the lower proportion drug is an antimuscarine bronchodilator.

33. A formulation as claimed in claim 27, wherein the higher proportion drug is a corticosteroid and the lower proportion drug is a short or medium acting beta agonist or an antimuscarine bronchodilator.

34. A formulation as claimed in claim 33, wherein the lower dose drug is selected from formoterol, salmeterol, bambuterol and salts thereof.

35. A formulation as claimed in claim 27, wherein the higher proportion excipient is a sugar.

36. A formulation as claimed in claim 35, wherein the excipient is selected from lactose, mannitol, dextrose, trehalose, xylitol, sorbitol and mixtures thereof.

37. A formulation as claimed in claim 1, including one or more stabiliser.

38. A method of manufacture of a formulation for pulmonary or nasal administration as claimed in any of preceding claim, including the step of making a mixture of particles of two or more drugs or excipients by spray drying without further processing of the particles.

39. A metered dose dry powder inhaler containing a powder reservoir containing a formulation in accordance with claim 1, optionally also containing one or more further drugs or excipients.

40. A metered dose dry powder inhaler containing generally spherical particles 90% of which have a dimension below 5 μm which have been produced by spray drying of a formulation as claimed in claim 1.

41. A capsule for insufflation containing generally spherical particles 90% of which have a dimension below 5 μm as claimed in claim 1.

42. A unit dose pocket or blister package containing generally spherical particles 90% of which have a dimension below 5 μm as claimed in claim 1.

43. A metered dose inhaler containing a CFC propellant selected from propellant P134a, P227 or a mixture thereof and a suspended formulation as claimed in claim 1.
